(19) United States
(12) Patent Application Publication
(10) Pub. No.: US 2026/0287478 A1
(43) Pub. Date: Sep. 24, 2026

(54) SYSTEM FOR SAMPLING GAS FROM A SOLID POROUS MEDIUM

(71) Applicant: ENGIE, Courbevoie (FR)

(72) Inventors: Maria ROSANNE, Courbevoie (FR); Serge JUNCA, Garlin (FR)

(21) Appl. No.: 19/476,349

(22) PCT Filed: Apr. 16, 2024
(86) PCT No.: PCT/FR2024/050504

§ 371 Date: Oct. 17, 2025

(30) Foreign Application Priority Data
Apr. 20, 2023 (FR) 2303985

(51) Int. Cl. G01N 1/22; G01N 33/00; G01N 33/24

(52) U.S. Cl. CPC G01N 1/2294; G01N 33/0009; G01N 33/24; G01N 2001/2285

(57) ABSTRACT

The present invention relates to a system for sampling gas from a porous medium, the system including a sampling chamber, an insertion end of which is intended to be pressed into the porous medium, which chamber is delimited by a gas-permeable casing and provided with an outlet opening; an outlet duct connected to the outlet opening and to an external medium; a pipe extending from a first end connected to the sampling chamber to a second end connected to an external medium through a control member that enables or prevents the passage of a gas flow; a suction pump for suctioning, through the outlet opening, a gas from the sampling chamber to the outlet duct.

### TECHNICAL FIELD OF THE INVENTION

The present invention relates to the general field of characterizing soil gas. More particularly, it relates to a system for sampling gas from a solid porous medium for analysis. This sampling system is designed to collect any type of gas trapped in a porous solid medium, such as hydrogen or helium, especially for analysis purposes.

### TECHNICAL BACKGROUND

In the context of the energy transition, there is growing interest in hydrogen.

For example, in the transportation sector, hydrogen appears to be a promising replacement for oil and its derivatives. When used as a fuel in an internal combustion engine combined with oxygen or as an energy carrier in a fuel cell, hydrogen substantially produces water and heat.

Most of the world's hydrogen is currently produced from hydrocarbons, in particular through the steam reforming of natural gas. While efficient, this technique produces more carbon dioxide per unit of heat than fossil fuel combustion. The challenge therefore lies in finding ecologically viable alternatives for hydrogen production that justify its use as a substitute for fossil fuels.

By chance, humans have observed hydrogen emanating from the surface of the Earth's crust. At this stage, the plan is to collect this spontaneous and passive output. Now that this production has been identified, the next step is to determine the so-called deposit locations, i.e., the richest areas for extraction.

To this end, the aim is to quantify the flow of hydrogen emanating from the soil through on-site sampling.

There are different types of gas analyzers. These analyzers present a sampling system and, typically, a measuring chamber equipped with sensors that measure the concentration of collected gas.

There are three main sampling systems for collecting the gas in the measuring chamber:
passive gas capture systems, which use diffusion and are presented in the form of bells positioned flush with the surface or probes inserted into the ground, trap gases diffusing from the ground;
pump systems, which commonly include a sampling tube intended to be inserted into the ground in communication with the measuring chamber and an internal pump to draw out gases trapped in the ground by suction; and
sampling systems configured to recover gas using a syringe, through a probe driven into the ground.

However, these systems are limited in their ability to accurately determine the volume flow rate, i.e., the flow of hydrogen generated by the soil.

When using a passive diffusion system, a gas concentration equilibrium is established between the porous media and the system's inner chamber, hindering the determination of hydrogen flow.

For a sampling system, only spot measurements are permitted. This means that regular monitoring over time is not possible, which is particularly disadvantageous given the potential for fluctuations in hydrogen flows due to various factors that may not occur daily or regularly. As understood, hydrogen flow determined in isolation cannot be considered representative of reality.

Active pump systems suction the gas that is trapped within the porous medium surrounding the sampling tube. This radius of action depends on a certain number of parameters, including permeability, diffusion coefficient, and variations in pressure and flow rate over time. As the pump operates, the porous medium is systematically drained of gas by the suction effect. This can result in pump asphyxiation due to a pressure drop, also known as “pump depletion”. Because the pump's radius of action varies unpredictably based on pressure variations, the volume of gas suctioned by the pump cannot be determined with any certainty. Since the volume of gas recovered in the measuring chamber cannot be defined, it is impossible to determine the hydrogen flow rate based on the measured concentration.

The aim of the invention is to overcome the previously identified disadvantages by providing an accurate gas sampling system for determining the flow of hydrogen emanating from porous soil.

### DESCRIPTION OF THE INVENTION

To this end, the subject matter of the invention is a system for sampling gas from a solid porous medium, especially for determining a flow rate of gas diffusing from said solid porous medium, the solid porous medium being bounded by a surface; the system comprising:
a sampling chamber, extending along a longitudinal axis, which is intended to be pressed at least partially into the porous medium by a longitudinal insertion end, which sampling chamber is delimited by a casing, a portion of which is gas-permeable to allow gas to diffuse from the solid porous medium into the sampling chamber, an outlet opening also being formed in the casing;
an outlet duct, one part of which is connected to an external medium and another part of which is connected directly or indirectly to the outlet opening;
a pipe extending from a first end connected to the sampling chamber to a second end connected to an external medium through a control member that makes enables or prevents the passage of a gas flow through this pipe;
a suction pump for suctioning, through the outlet opening, a gas from the sampling chamber to the outlet duct;

wherein, along the longitudinal axis, the first end of the pipe is closer to the insertion end than the opening is.

The invention also relates to a gas sampling system as defined above, wherein the external medium to which the outlet duct and the second end of the pipe are connected corresponds to:
the external atmospheric medium, or
gaseous reservoirs at iso-pressure, the outlet duct and the second end each being connected to a reservoir.

The invention also relates to a gas sampling system thus defined, wherein:
the first end of the pipe is adjacent to the insertion end; and
the outlet opening, formed in the casing of the sampling chamber, is adjacent to a longitudinal end of the sampling chamber which is opposite the insertion end.

The invention also relates to a gas sampling system thus defined, wherein the pipe includes a section that comprises the first end, which is centered on the longitudinal axis and extends within the sampling chamber.

The invention also relates to a gas sampling system thus defined, wherein the pipe extends outside the casing and is connected to the sampling chamber at its first end.

The invention also relates to a gas sampling system thus defined, comprising a connecting duct which connects the outlet opening to the outlet duct, the pump being interposed in this duct.

The invention also relates to a gas sampling system thus defined, wherein the control member is a solenoid valve.

The invention also relates to a gas analyzer for determining an hourly volume flow rate of gas diffusing from a solid porous medium, comprising a sampling system thus defined, and an instrumented measuring chamber, equipped with a gas concentration measurement sensor, this measuring chamber being connected to the sampling chamber and to the outlet duct.

The invention also relates to a method for measuring the concentration of a gas diffusing from a solid porous medium by means of an analyzer thus defined, the sampling chamber of which is pressed into the solid porous medium by its insertion end, the method comprising the successive steps of:
a) switching off the pump and actuating the control member to prevent the passage of a gas flow through the pipe, the gas diffusing passively from the porous medium into the sampling chamber;
b) activating the control member to enable a gas flow through the pipe and starting the pump to pump the gas from the sampling chamber to the measuring chamber;
c) measuring the concentration of the gas collected in the measuring chamber using the sensor.

The invention also relates to a method thus defined, wherein the pumping time in step b) is predefined so that the pump stops before the gas is discharged through the outlet duct via the action of the pump.

The invention also relates to a method thus defined, further comprising a preliminary step a′) of initiating the diffusion of the gas, wherein the control member is actuated to prevent the passage of a gas flow through the pipe and the pump is switched on.

### BRIEF DESCRIPTION OF THE DRAWINGS

Further features and advantages of the invention will become apparent from the following detailed description, which may be understood with reference to the attached drawings in which:

FIG. 1 is a schematic depiction of a gas analyzer according to the invention, comprising a measuring chamber equipped with sensors and a pump sampling system for collecting and transferring gas to the measuring chamber; this system comprising a sampling chamber, a pipe which is open to the external medium and which opens into the sampling chamber, a control member for enabling or preventing the passage of an air flow along the pipe, and a pipe for recovering gas from the sampling chamber.

FIG. 2 is a partial sectional view of an embodiment of the sampling system wherein the sampling chamber has a tubular architecture and the pipe extends partially coaxially into this sampling chamber;

FIG. 3 is a detailed view of FIG. 2;

FIG. 4a is a schematic depiction of the gas analyzer shown in FIG. 1, illustrating an arrangement of the pump and the control member during a step of passive gas diffusion from the porous medium into the sampling chamber.

FIG. 4b is a schematic representation of the gas analyzer shown in FIG. 1, illustrating a step in which the gas contained in the sampling chamber is pumped to the measuring chamber;

FIG. 4c is a schematic view of the gas analyzer shown in FIG. 1, illustrating a depletion step if the configuration of an active pump system is desired.

FIG. 5 is a schematic representation of a variant of the gas analyzer characterized by a different pipe arrangement.

### DETAILED DESCRIPTION OF THE INVENTION

To describe the invention and understand the claims, a non-limiting vertical Z direction linked to the Galileo terrestrial reference frame will be adopted with the longitudinal and radial AX and AY axes linked to the gas analyzer.

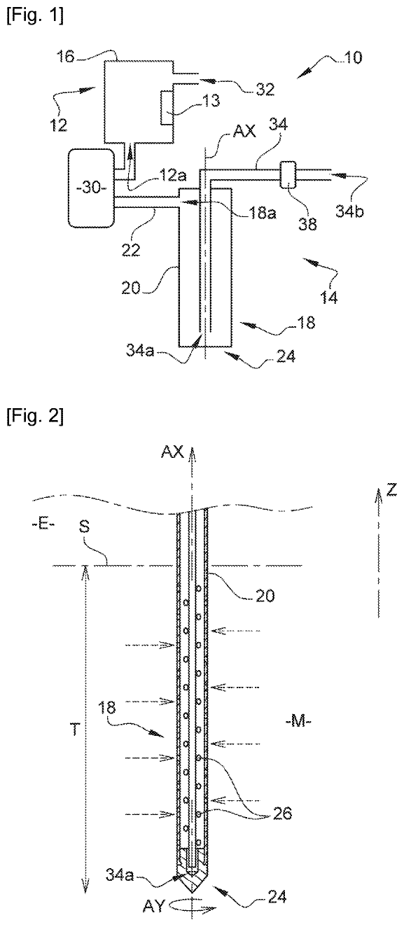

Referring to FIGS. 1 to 3, a gas analyzer according to the invention, marked 10, conventionally comprises a measuring chamber 12 and a gas sampling system 14.

Hereinafter, analyzer 10 will be disclosed for an example application of measuring natural hydrogen emanation in a porous medium. However, the analyzer 10 is not limited to this particular application, and can be used to collect and analyze any type of gas emanating from a porous medium.

The measuring chamber 12 is delimited by a casing marked 16. It encloses a set of sensors, especially a hydrogen concentration sensor, a temperature sensor, and a humidity sensor, designed to characterize the gas inside the casing. The sensors are collectively referred to as 13. Advantageously, this measuring chamber 12 presents a parallelepiped configuration so that it can be fitted with other sensors as required on these different faces.

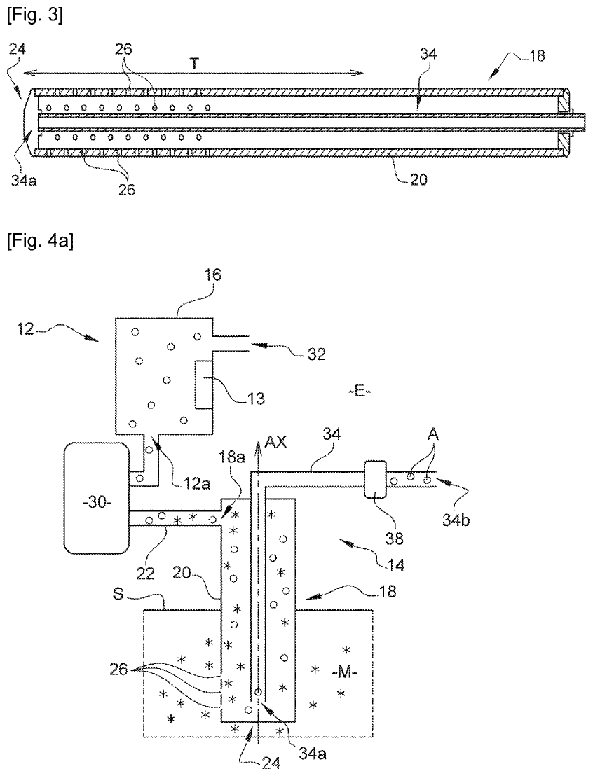

The gas sampling system 14 comprises a sampling chamber 18 designed to collect gas emanating from or trapped in a porous solid medium M. In the example shown in FIG. 2, this medium M is defined by a surface S which separates it from the ambient external medium E.

This chamber, delimited by a casing 20, communicates with the measuring chamber 12 by means of a connecting duct 22 of the sampling system 14. As understood, this connecting duct 22 is used to provide a pathway for the gas collected in the sampling chamber 18 to the measuring chamber 12 for analysis.

In the example shown in the figures, this connecting duct 22 connects openings 12a and 18a formed respectively in the casing of the measuring chamber 12 and in the casing of the sampling chamber 18.

The sampling chamber 18 is especially intended to be pressed into the porous solid medium M. Advantageously, the casing 20 has a tubular shape with a central longitudinal revolution axis AX. This particular morphology allows the sampling chamber 18, also known as a “sampling tube,” to penetrate the solid medium M.

It is preferred to orient the gas analyzer 10 so that the central axis AX is substantially coinciding with the vertical direction Z, but the invention is not strictly limited to this particular orientation. It can be positioned in a different orientation, for example at an angle to the vertical Z direction.

In practice, pressing the sampling chamber 18 into the porous solid medium M consists of:
positioning one longitudinal end of the sampling chamber 18, known as the insertion end marked 24, facing the surface S of the porous solid medium M; and
exerting inward pressure on the solid medium M until the sampling chamber 18 is pressed into this porous medium M along a section T of predefined length, measured from the insertion end 24.

To collect the gas from the porous medium M in the sampling chamber 18, its casing 20 is shaped so as to be permeable to gas along all or part of its section, marked T, intended to be pressed into the porous solid medium M. This arrangement allows the hydrogen contained in the porous medium M to diffuse into the sampling chamber 18.

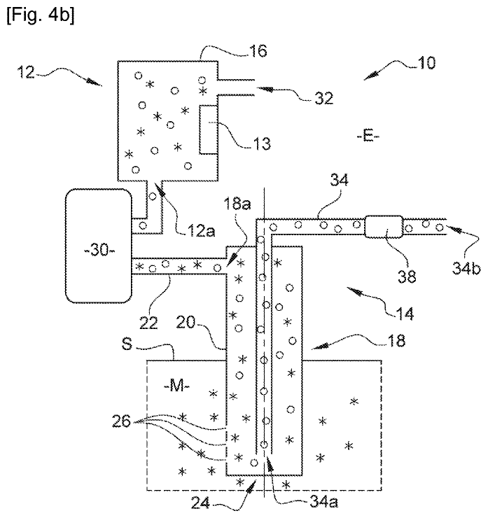

In the example shown in FIGS. 2 and 3, the envelope includes holes 26 formed through the casing 20 for this purpose. Hydrogen diffusion from the porous solid medium M to the sampling chamber 18 is illustrated by arrows.

These holes 26 are advantageously distributed at regular intervals along the longitudinal axis AX and radially about this axis along AY, so as to sweep the field uniformly about the section T. Note that an arrangement wherein the holes 26 are distributed differently may be selected. The wall 20 may, especially, be without holes 26 over a specific length in the vicinity of the insertion end 24.

It should be noted that the invention is not limited to the formation of such holes 26 to enable a passage of gas to the sampling chamber 18. These holes 26 may, especially, be replaced by a gas-permeable membrane without departing from the scope of the invention.

In addition, the gas sampling system 14 comprises a pump 30 designed to suction the gas contained in the sampling chamber 18 through the outlet opening 18a and transfer it to the measuring chamber 12. In the example shown in the figures, this pump 30 is interposed in the duct 22, at the interface between the sampling chamber 18 and the measuring chamber 12. It should be noted that a different location for the pump 30 can be selected, for example downstream of the measuring chamber 12.

The idea behind the invention is to reliably determine the gas flow emanating from the porous solid medium M. On this basis, the aim is to be able to operate the pump without depletion.

In this respect, the gas sampling system 14 is configured to allow pressure equilibrium while the pump 30 is operating. In the example shown in the figures, this is achieved by keeping the measuring chamber 12 and the sampling chamber 18 in communication with the external atmospheric medium E while the pump 30 is operating.

In the example shown in FIGS. 1 to 3, the gas sampling system 14 comprises:
an outlet duct 32, or vent, formed in the extension of the measuring chamber 12, which opens onto the external atmospheric medium E; and
a pipe 34 which extends from a first end 34a connected to the sampling chamber 18 to a second end 34b which opens out into the external atmospheric medium E.

As understood, the outlet duct 32 and the pipe 34 are arranged to ensure communication with the external atmospheric medium E when the sampling chamber 18 is pressed into the porous solid medium M.

In the example shown in FIGS. 1 to 3, the pipe 34 extends from the first end 34a, advantageously positioned in the sampling chamber 18. More specifically, a section of the pipe, which comprises the first end 34a, extends coaxially along the sampling chamber 18. This pipe 34 has a smaller cross-sectional diameter than the sampling chamber 18, so that it extends within this chamber.

From a dimensional point of view, according to a preferred embodiment of the invention:
the length of the sampling chamber 18 and the pipe 34 can vary from 0.8 to 3 m;
the internal diameter of the pipe 34 is set between 4 and 7 mm and the internal diameter of the sampling chamber 18 is set between 9 and 12 mm;
the holes 26 can be between 0.5 and 2.5 mm in diameter, distributed over a length of between 40 mm and 150 mm. They can especially be spaced between 4 and 15 mm apart.

The second end 34b of the pipe 34 and the outlet duct 32 thus consist of the inlet and outlet respectively of a gas circuit under the action of the pump 30. With this solution, the aim is to drain the sampling chamber 18, into which hydrogen has diffused, into the measuring chamber 12 by introducing external ambient air into the sampling chamber 18 via the pipe 34, which allows work at atmospheric pressure at all times.

In practice, this external air, introduced by the pipe 34 into the sampling chamber 18, pushes the gas, initially contained in this chamber, towards the measuring chamber 12.

To ensure controlled movement of the gas initially contained in the sampling chamber 18 towards the measuring chamber 12, the first end 34a of the pipe and the so-called outlet opening 18a are arranged in a particular way, to which the pipe 22 connecting the sampling chamber 18 to the measuring chamber 12 is connected.

As part of this hydrogen collection application, its volatile nature in the air must be taken into account when designing the gas sampling system 14. Hydrogen is inherently less dense than ambient air. On this basis, to ensure that the gas initially contained in sampling chamber 18 is functionally drained, it is necessary to introduce ambient air vertically below the outlet opening 18a. Otherwise, the ambient air traveling along the pipe 34 would likely rush directly into the duct 22 once it reaches the sampling chamber 18 under the action of the pump

In order to achieve controlled gas displacement, the first end 34a of the pipe 34 must be positioned structurally closer to the insertion end 24 than to the outlet opening 18a.

In practice, to achieve complete and controlled emptying of the sampling chamber 18, it is necessary to:
introduce ambient air into the sampling chamber as close as possible to the insertion end 24, and
form the outlet opening 18a at the opposite longitudinal end of the sampling chamber.

In other words, this involves arranging the first end 34a of the pipe 34 adjacent to the insertion end 24, and further arranging the outlet opening 18a adjacent to the longitudinal end of the sampling chamber 18 which is opposite the insertion end.

With this arrangement, ambient air can fill the sampling chamber 18 under the action of the pump 30 as it gradually rises from the first end 34a of the pipe. This has the effect of simultaneously driving the gas initially contained, i.e., diffused hydrogen, towards the measuring chamber 12.

Finally, the gas sampling system 14 further comprises a control member 38 for enabling or preventing the passage of ambient air along the pipe 34. This control element 38 may take the form of a set-point solenoid control valve, but the invention is not limited to this feature. By way of example, this control member 38 may take the form of a quarter-turn valve.

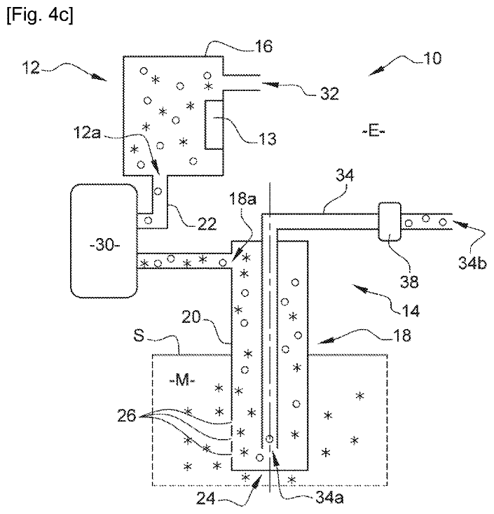

Referring to the attached FIGS. 4a to 4c, the method for measuring the concentration of hydrogen diffusing from the solid porous medium M using the analyzer 10 according to the invention is described. For illustration purposes, hydrogen and air are depicted in these figures by star-shaped and round symbols respectively.

Once the sampling chamber 18 has been pressed into the solid porous medium M by its insertion end 24, the first step is to allow the hydrogen to diffuse passively into this sampling chamber. To this end, the pump 30 is switched off and the control member 38 is actuated so as to prevent any atmospheric air flow through the pipe 34. As understood, the purpose of blocking the pipe 34 with the control member 38 is to prevent diffused hydrogen from traveling unintentionally along the pipe 34 until it is released into the atmosphere. Such a hydrogen leak would result in a loss and, consequently, lead to a falsely measured concentration.

After a predefined waiting time, the process requires the gas now contained in the sampling chamber 18 to be transferred to the measuring chamber 12. To achieve this, the control member 38 is actuated to enable air to flow along the pipe 34 and, at the same time, the pump 30 is switched on. The pump 30 creates an internal gas flow between the second end 34b of the pipe and the outlet duct 32. A suction effect is created at the outlet opening 18a, leading to the penetration of gas from the sampling chamber 18 into the measuring chamber 12, simultaneously with the introduction of ambient air into the sampling chamber 18 via the pipe 34. This results in the gas produced by diffusion being moved towards the measuring chamber.

In practice, the pump is actuated to stop pumping before the gas is discharged through the outlet duct 32. As the pump operates without depletion, its operating speed does not vary during pumping, in other words, its effective flow rate is constant. Therefore, it is possible to limit the time of its actuation to just what is necessary to convey a volume of gas that will occupy the predefined volume capacity of the measuring chamber 12.

The next step consists of collecting the sensor acquisition results, in particular recording the measured hydrogen concentration. On this basis, the measuring chamber 12 acts as a storage airlock while the sensors measure the hydrogen concentration, temperature, and relative humidity of the gas. Given that the volume of gas traveling into the measuring chamber is known and, in the absence of pump depletion, the specific volume of hydrogen and its hourly flow rate can be calculated directly from the measured concentration value.

Alternatively, the pump can be operated for random periods of time. In this case, the gas cannot remain in measuring chamber 12 because it is expelled into the external medium E through the outlet duct 32. This gas can especially be collected directly from the outlet duct 32 using a receptacle, such as a bag, for subsequent measurements, for example, using chromatography to identify the elements in the collected gas or spectrometry for measuring isotopic composition.

The method according to the invention can further comprise a preliminary step upstream of the aforementioned diffusion step. In practice, when the diffusion coefficient in solid porous medium M is low, it may be desirable to speed up the filling of the sampling chamber 18 through the sampling tube before the next measurement.

This optional step requires the control member 38 to block the pipe 34 and start the pump 30. As understood, the pump will then operate in depletion during this stage, in the configuration of an active pump system. It should be noted, however, that this step is designed to be sufficiently short to prevent the flow rate of the pump 30 from dropping to zero.

Concretely, this step corresponds to a step of initiating gas diffusion, aimed at forcing the emanation of hydrogen from the solid porous medium M.

The invention thus relies on both gas diffusion and the use of a pump, without however being subjected to a state of depletion, the disadvantages of which have been identified. In this way, the invention makes it possible to determine with certainty a value for hourly volume flow, in other words, the flow of hydrogen that has diffused, and thus help to assess the opportunity for this particular application.

Notwithstanding the reliability of the characterization achieved by the analyzer 10 according to the invention, avoiding a depletion effect in accordance with the invention enables locally-spaced sampling. Indeed, depletion leads to imbalance in the solid porous medium M with respect to the trapped gases, and also disturbs the equilibrium with the neighboring medium. This may be a disadvantage when the intention is to place an array of closely spaced analyzers on a given surface to determine the total flow rate for an area of interest.

In the example shown in FIGS. 1 to 3, the pipe 34 is described as extending into the sampling chamber 18. This arrangement effectively allows the sampling chamber 18 to penetrate the porous solid medium without being obstructed.

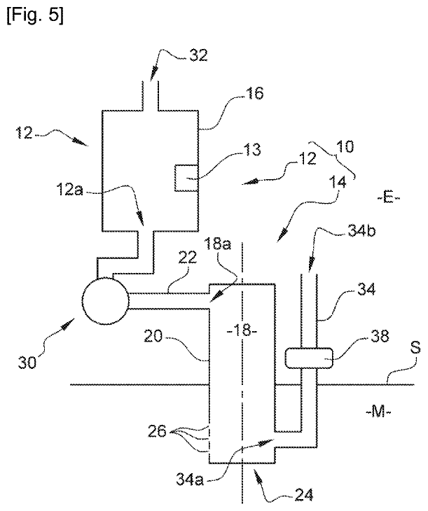

Nevertheless, the invention allows for an arrangement wherein the pipe extends outside the sampling chamber. This embodiment, illustrated in FIG. 5, differs from that described with reference to FIGS. 1 to 3 in the arrangement of the pipe 34. It is connected to the sampling chamber 18 by its first end 34a, opposite an opening formed for this purpose in the casing 20.

According to this embodiment, the structural condition is always met in that the first end 34a of the pipe 34 is arranged closer to the insertion end 24 than it is to the outlet opening 18a.

In addition, the gas sampling system 14 has been explained as avoiding pump depletion by communicating with the external atmospheric environment, at the inlet and outlet of the pumping circuit consisting of the second end 34b of the pipe and the outlet duct 32, respectively. Note that the invention is not limited to this particular arrangement.

In practice, a depletion effect is observed when there is a pressure differential. On this basis, an arrangement can be selected wherein the second end 34b of the pipe and the outlet duct 32 are connected to pressure-controlled reservoirs. As long as these reservoirs are configured to achieve identical pressures, iso-pressure pump operation between the inlet and outlet ensures the same effect as a connection to the external atmospheric medium.

As understood, the external medium with which the outlet duct 32 and the pipe 34 communicate is not limited to the atmospheric external medium E, as has been described on the basis of the appended figures. In practice, the outlet duct 32 and the pipe 34 can be connected to a common ambient medium or each to a separate medium, as long as a similar pressure at the inlet and outlet of the pump circuit is maintained, either passively or by control means.

The gas sampling system 14 has been described as an integral part of the analyzer 10. The analyzer is characterized by the addition of the measuring chamber 12 to the gas sampling system 14. It should be noted that the gas sampling system 14 can be considered in isolation, by substituting the measuring chamber 12 with a removable receptacle, the contents of which can be analyzed in due course. In the absence of a measuring chamber 12, the outlet duct 32 can be formed directly by the duct 22, and the pump 30 can be interposed in this duct 22. Generally speaking, the outlet duct 32 can thus be broadly defined as comprising a part which is connected to the external medium and another part of which is connected directly or indirectly to outlet opening 18a.

The sampling chamber 18 is best described as having a tubular, in other words, cylindrical, contour. The invention is not limited to this particular arrangement, and allows for any type of shape. In particular, a variable-geometry sampling chamber, for example telescopic, can be selected, enabling its dimensions to be varied to adapt to a particular diffusion volume, or to accommodate sampling at a desired depth.

Finally, the invention has been described in an application for measuring the emanation of natural hydrogen in a porous medium, making it possible to identify particularly favorable exploitation sites. It should be noted that the device is not limited to this particular application. In practice, it can be used to assess the diffusion of any type of gas in a porous medium.

In particular, the relative positioning of the first end 34a of the pipe 34 and the outlet opening 18a with respect to the insertion end 24 as defined for collecting hydrogen also remains applicable for any gas that is less dense than ambient air.

Note that in the case whereupon the second end 34b of the pipe and the outlet duct 32 are connected to reservoirs, these reservoirs are not limited to containing air. They may contain another gaseous fluid, which may in particular be selected according to its density.

The content of several components can especially be analyzed at the same time, using suitable measuring sensors. In this respect, if a membrane is used in place of the holes 26 formed in the casing 20 of the sampling chamber, the latter can be selectively permeable to one or more types of gas (dense, corrosive, toxic, explosive). In the case of corrosive gases, the materials making up the analyzer 10 must be adapted to the type of corrosion, or else protected by a suitable coating.

The range of possible applications includes, but is not limited to:
detecting gas leaks for infrastructure safety;
quantifying the volume of radon, helium, H2S, or various pollutants such as volatile organic compounds; or
monitoring geological deposits or monitoring gas emissions, such as sulfur, to monitor volcanic activity.

1. A system for sampling gas from a solid porous medium, especially for determining a flow rate of gas diffusing from said solid porous medium, the solid porous medium being bounded by a surface,
the system comprising:
a sampling chamber, extending along a longitudinal axis, which
is intended to be pressed at least partially into the solid porous medium by a longitudinal insertion end, which sampling chamber is delimited by a casing, a portion of which is gas-permeable to allow gas to diffuse from the solid porous medium into the sampling chamber, an outlet opening also being formed in the casing;
an outlet duct, one part of which is connected to an external medium and another part of which is connected directly or indirectly to the outlet opening;
a pipe extending from a first end connected to the sampling chamber to a second end connected to an external medium through a control member that enables or prevents passage of a gas flow through this pipe;
a suction pump for suctioning, through the outlet opening, a gas from the sampling chamber to the outlet duct;
wherein:
along the longitudinal axis, the first end of the pipe is closer to the insertion end than the outlet opening is.

2. The system according to claim 1, wherein the outlet duct and the second end of the pipe are connected to the external medium or to gaseous reservoirs which are maintained at iso-pressure.

3. The system according to claim 1, wherein:
the first end of the pipe is adjacent to the insertion end; and
the outlet opening, formed in the casing of the sampling chamber, is adjacent to a longitudinal end of the sampling chamber which is opposite the insertion end.

4. The system according to claim 1, wherein the pipe includes a section that comprises the first end, which is centered on the longitudinal axis and extends within the sampling chamber.

5. The system according to claim 1, wherein the pipe extends outside the casing and is connected to the sampling chamber at its first end.

6. The system according to claim 1, comprising a connecting duct which connects the outlet opening to the outlet duct, the suction pump being interposed in this duct.

7. The system according to claim 1, wherein the control member is a solenoid valve.

8. A gas analyzer for determining an hourly volume flow rate of gas diffusing from a solid porous medium, comprising a sampling system according to claim 1, and an instrumented measuring chamber, equipped with a gas concentration measurement sensor, this measuring chamber being connected to the sampling chamber and to the outlet duct.

9. A method for measuring the concentration of a gas diffusing from the solid porous medium by means of the gas analyzer according to claim 8, the sampling chamber of which is pressed into the solid porous medium by its insertion end, the method comprising the successive steps of:
a) switching off the suction pump and actuating the control member to prevent the passage of a gas flow through the pipe, the gas diffusing passively from the porous medium into the sampling chamber;
b) activating the control member to enable a gas flow through the pipe and starting the suction pump to pump the gas from the sampling chamber to the measuring chamber;
c) measuring the concentration of the gas collected in the measuring chamber using the sensor.

10. The method according to claim 9 wherein a pumping time in step b) is predefined so that the suction pump stops before the gas is discharged through the outlet duct via action of the suction pump.

11. The method according to claim 9, further comprising a preliminary step a′) of initiating the diffusion of the gas, wherein the control member is actuated to prevent the passage of a gas flow through the pipe and the suction pump is switched on.

12. The method according to claim 10, further comprising a preliminary step a′) of initiating the diffusion of the gas, wherein the control member is actuated to prevent the passage of a gas flow through the pipe and the suction pump is switched on.
